(19) United States
(12) Plant Patent Application Publication
(10) Pub. No.: US 2016/0198607 P1
(43) Pub. Date: Jul. 7, 2016

(54) Hosta plant named 'Ambrosia'

(50) ;

(71) Applicant: Robert M. Solberg, Franklinton, NC (US)

(72) Inventor: Robert M. Solberg, Franklinton, NC (US)

(21) Appl. No.: 14/544,414

(22) Filed: Jan. 5, 2015

(51) Int. Cl. A01H 5/00

(52) U.S. Cl. PLT/353

(57) ABSTRACT

A new and distinct cultivar of Hosta named ‘Ambrosia, characterized by its large sized mounding clump, blue leaf margin with a light yellow center, the presence of a heavy glaucous wax over the entire leaf surface, vigorous growth rate, and pale lavender fragrant flowers. In combination these traits set ‘Ambrosia’ apart from all other existing varieties of Hosta known to the inventor.

### BACKGROUND OF THE INVENTION

The present invention relates to a new and distinct cultivar of Hosta plant, botanically known as Hosta, hereinafter referred to by the cultivar name ‘Ambrosia’.

The new plant was discovered by the Inventor during the summer of 2008 as a non-induced, naturally occurring whole plant mutation of Hosta ‘Guacamole’ (not patented) at Pender Nursery in Garner, N.C., USA. Asexual propagation of the new cultivar by division in 2011 in Franklinton, N.C. and by tissue culture in 2012 in Olathe, Kans. has shown the unique and distinct characteristics of this new plant are stable and reproduce true to type in successive generations.

### SUMMARY OF THE INVENTION

The following traits have been repeatedly observed and are determined to be the unique characteristics of ‘Ambrosia. These characteristics in combination distinguish the new Hosta as a new and unique cultivar:

1. Large sized mounding clump;

2. Blue leaf margin with a light yellow center;

3. The presence of a heavy glaucous wax over the entire leaf surface;

4. Vigorous growth rate; and

5. Pale lavender fragrant flowers.

The new Hosta can be compared to its parent cultivar, Hosta ‘Guacamole’. In the new Hosta, the leaf margin is blue due to the presence of a glaucous wax as compared to the dark green margin of Hosta ‘Guacamole’ which has no wax. The leaf center of the new Hosta is a light yellow due to the presence of a glaucous wax as compared to the yellow/chartreuse center of Hosta ‘Guacamole’ which has no wax.

The new Hosta cultivar has not been observed under all possible environmental conditions. The phenotype may vary to some extent with variations in environmental conditions such as temperature, fertility and light intensity, but without any variance in the genotype.

### BRIEF DESCRIPTION OF THE PHOTOGRAPHS

The accompanying color photographs illustrate the overall appearance of the new cultivar including its unique traits as a 5 year old plant growing in a three gallon container. The colors are as true as is reasonably possible with conventional photography.

FIG. 1 was taken in May and comprises a top perspective of an individual leaf of the new Hosta.

FIG. 2 was taken in May and shows the entire Hosta clump.

FIG. 3 was taken in August and shows the Hosta flower scape.

FIG. 4 was taken in May and compares a Hosta clump of ‘Ambrosia’ (right) with ‘Guacamole’ (left)

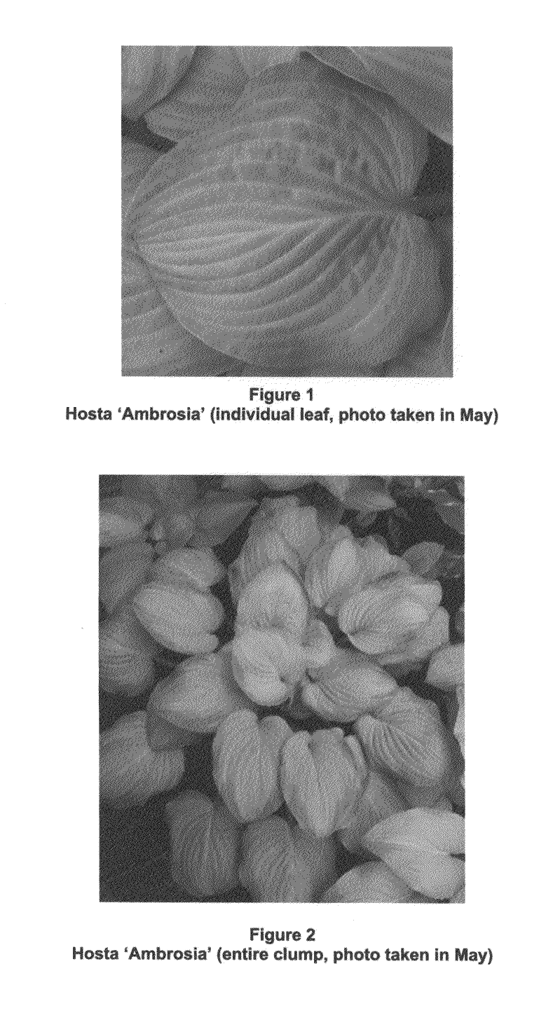

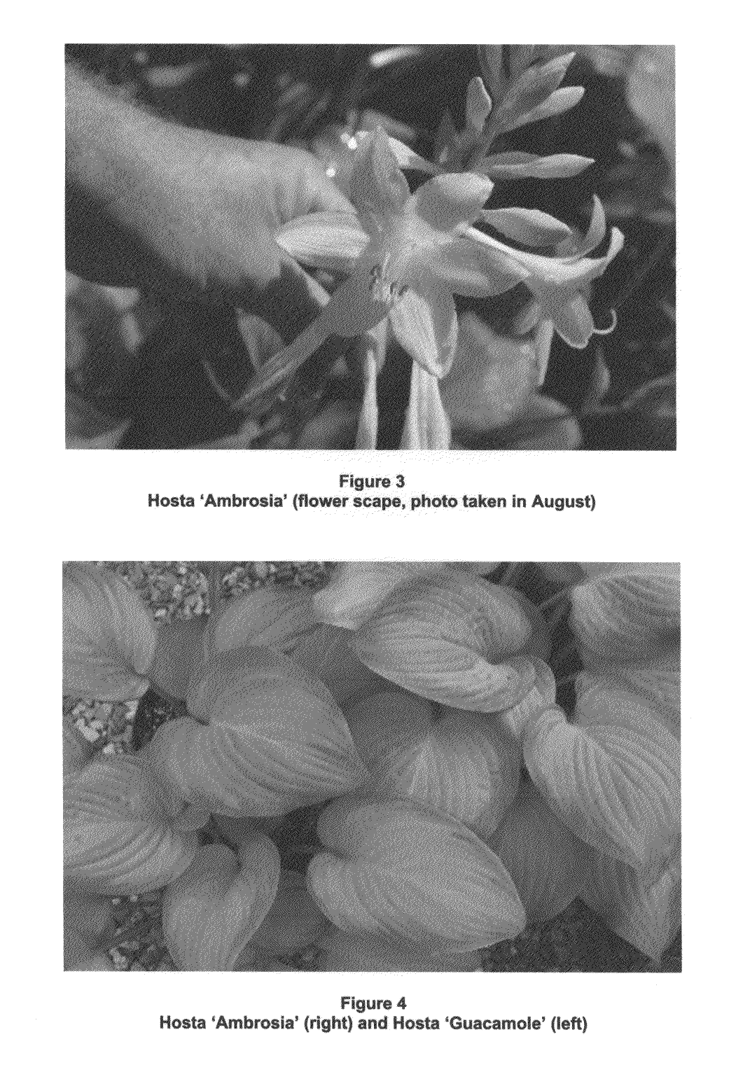

### DETAILED DESCRIPTION OF THE INVENTION

In the following description, color references are made to The Royal Horticultural Society's Colour Chart (2001 edition) except where general terms of ordinary dictionary significance are used. The following observations and measurements describe a 5-year old plant growing in a three gallon container, as depicted in the accompanying photographs, which was grown outdoors in Franklinton, N.C., USA.
Botanical classification: Hosta ‘Ambrosia’.
Parentage: Naturally occurring whole plant mutation of Hosta ‘Guacamole’ (non patented).
Propagation:
   Method.—By division and tissue culture.
Plant description:
   Plant habit.—Compact, mounding, symmetrical.
   Culture.—Light to medium shade in moist, well-drained soil.
   Plant type.—Herbaceous rhizomatous perennial.
   Plant height.—24 inches.
   Plant width.—54 inches.
   Growth rate.—Vigorous.
   Root system.—Normal, fleshy, branching from central rhizome.
   Disease resistance.—No known resistance or susceptibility to disease known to Hosta has been observed.
Foliage description: Leaf shape — Ovate with a cordate base. Leaf margin — Entire. Leaf surface — Smooth, very glaucous on top and bottom. Leaf texture — Moderate substance, slightly wavy leaf margins. Leaf dimensions — 8½ inches in width, 11 inches in length. Venation pattern — Campylodrome with 12 to 13 pairs of veins. Leaf color — Light yellow center (about RHS 3D) with a blue margin (about RHS 123C). The leaf center starts the growing season light yellow (about RHS 3D) and later darkens to yellowish green (about RHS 142A). The leaf margin starts the growing season blue (about RHS 123C) and later darkens to brilliant green (about RHS 129A). The leaf margin width is about ⅝ inches.
Petiole description: Plant petioles have a length of approximately 16 to 20 inches with a diameter of about ½ to ¾ inches. The petiole has a central region with a light yellow color (about RHS 3D) with a margin having a blue color (about RHS 123C).
Flower description:
   Bloom period.—Mid-summer (late July-August in Franklinton, N.C.).
   Fragrance.—Strongly fragrant, sweet, pleasant.
   Flower arrangement.—Raceme of numerous single flowers comprised of six tepals. The number of flowers per raceme varies from about 25 to 30 with each flower lasting approximately one day over a period of about 3 weeks.
   Flower shape.—Funnel-shaped.
   Flower dimensions.—3 inches in length and 2¼ inches in width.
   Flower color.—Pale lavender (about RHS 76D).
   Bud color.—Pale lavender (about RHS 76C).
   Bud dimensions.—2¼ inches in length and ⅝ inches in width.
   Tepal color.—inner pale lavender (about RHS 76D); outer pale lavender (about RHS 76D).
   Tepal dimensions.—1⅝ inches in length and ⅝ inches in width.
   Bract color.—Yellowish green (about RHS 142A).
   Bract length.—1⅛ to 1½ inches.
   Flower scape length.—30 to 41 inches tall.
   Flower scape color.—Yellowish green (about RHS 142A).
   Flower scape angle.—Arching 30 degrees from vertical.
   Pedicel color.—Pale lavender (about RHS 76D).
   Pedicel dimensions.—⅜ inches in length and 3/16 inches in width.
   Raceme length.—20 to 22 inches from lowest flower to top of inflorescence.
Scape description:
   Number.—The number of scapes per plant is dependent on the maturity of the plant. Each mature eye comprising the clump may produce a single flower scape under normal growing conditions.
Description of reproductive organs: The reproductive organs are typical of the genus Hosta and comprise six stamens and a compound ovary having three locules. The stamen color is pale lavender (about RHS 76D); the anther color is moderate purple (about RHS 79D); the pistil color is pale lavender (about RHS 76D); and the stigma color is pale lavender (about RHS 76D). The stamen length is about 2½ to 3 inches and the pistil length is about 3½ inches, curved 180 degrees at the end.
Seed development: Has not been observed.
Root development: From transfer to rooting media in tissue culture, rooting takes approximately 4 weeks at about 68 degrees Fahrenheit. After transfer from stage III in tissue culture to planting into soil in a greenhouse, a well rooted plant is produced in approximately 8 weeks with a daytime temperature of about 68 degrees Fahrenheit and a soil temperature of about 78 degrees Fahrenheit.

I claim:

1. A new and distinct cultivar of Hosta plant named ‘Ambrosia’ as illustrated and described herein.
